(19) United States
(12) Patent Application Publication
(10) Pub. No.: US 2011/0295615 A1
(43) Pub. Date: Dec. 1, 2011

(54) POST-SURGICAL ACCESSORY CONVENIENCE KIT

(76) Inventor: BRADLEY P. BENGTSON, Grand Rapids, MI (US)

(21) Appl. No.: 13/207,048

(22) Filed: Aug. 10, 2011

Related U.S. Application Data
(63) Continuation-in-part of application No. 12/288,255, filed on Oct. 17, 2008, now abandoned.
(60) Provisional application No. 60/999,328, filed on Oct. 17, 2007.

(51) Int. Cl. G06Q 50/00; A61F 15/00

(52) U.S. Cl. 705/2; 206/570; 602/48

(57) ABSTRACT

A post-surgical accessory convenience kit, or PACK, and associated devices and methods that allows communication of a patient's post-operative needs and regimens. The PACK and associated devices allows monitoring and remote transmitting of a patient's needs and regimens.

### RELATED APPLICATIONS

This application is a continuation-in-part of U.S. Ser. No. 12/288,255, filed on 17 Oct. 2008, which claims the benefit of U.S. Provisional Patent Application Ser. No. 60/999,328, filed 17 Oct. 2007, now abandoned.

### FIELD OF THE INVENTION

This application relates generally to the drainage of fluid from the body during the wound healing process, e.g., following surgery, trauma, or placement of implants or surgical devices, and, more particularly, to a post-surgical, post-operative kit sized to be worn by a patient to carry items that support their post-surgical needs.

### BACKGROUND OF THE INVENTION

Surgical drains are utilized in approximately 50% of major surgical procedures. Drains are required in many patients because the body cannot absorb the extra blood and fluid that occurs following many surgical procedures. A representative prior art continuous drain system can comprise an implanted device such as a piece of rubber tubing (Penrose drain) (as shown in FIG. 1), which provides dependent gravity drainage or responds to a negative suction force generated by a manual closed suction bulb. These types of drains constitute the most common devices currently available.

Drains, when they are used, are typically painful at the point they exit the skin, and can easily pull at these exit points. The suction bulbs that collect the fluid are typically attached to clothing with a safety pin or placed in a pocket, but commonly the tubing or bulb can get caught or pull at the skin or even be pulled out accidentally. As a result, patients do not like drains.

There is a need for a post-surgical, post-operative pack, not for any instruments, but for those items necessary for a patient's comfort, convenience, and post-surgical needs.

### SUMMARY OF THE INVENTION

The invention provides a post-surgical accessory convenience kit (in shorthand PACK). The kit is well suited for, but not limited to, serving the needs of patients who require drains. The kit includes devices that allow the patient to monitor treatment regimens, including conveying information for those treatment regimens remotely to and from the patient to a caregiver.

One aspect of the invention provides a kit including a reservoir, a length of tubing, and a pouch. The length of tubing has a first end adapted to be coupled to the reservoir and a second end adapted to be implanted into a drain site in a patent.

An additional aspect of the invention is a kit including dressing change materials.

An additional aspect of the invention is a kit including post-operative medication. The post-operative medication includes at least one of the following: pain medication, antibiotics, and/or ointments.

An additional aspect of the invention is a kit including at least one set of post-operative instructions.

An additional aspect of the invention is a kit including at least one drain record.

An additional aspect of the invention is a kit including at least one physician contact phone number.

An additional aspect of the invention is a kit including a pouch that is made of a material that is water and fluid resistant.

An additional aspect of the invention is a kit including a pouch made of a material that is clear.

An additional aspect of the invention is a kit including a pouch including a strap. In one embodiment the strap is sized and configured to secure the pouch around the waist of a patient. In an alternative embodiment the strap is sized and configured to wear the pouch over the shoulder of a patient.

An additional aspect of the invention is a kit including a pouch containing at least one pocket. An additional aspect of the invention is a kit wherein at least one of the pockets on the pouch includes a closure element.

An additional aspect of the invention is a kit including at least one auxiliary case attached to the outside of the pouch.

An additional aspect of the invention is a method for providing post-operative materials to a patient. The method may include providing a pouch with a plurality of compartments, providing a reservoir, providing tubing, providing post-operative medications, providing post-operative information, providing dressing change materials. The method may further include arranging the reservoir, tubing, post-operative medications, post-operative information, and dressing change materials within the compartments of the pouch. The method may further include distributing the pouch to a patient.

An additional aspect of the invention is a kit including a reservoir, a length of tubing, dressing change materials, post-operative medication, post-operative care instructions, physician contact phone numbers; and drain records. The length of tubing may have a first end adapted for coupling to the reservoir and a second end adapted for implanting into a drain site in a patient.

An additional aspect of the invention is a kit including a pouch, the pouch having a plurality of compartment.

An additional aspect of the invention is a kit wherein each of the items, including the reservoir, dressing change materials, post-operative medication, post-operative care instructions, physician contact phone numbers, and drain records are contained within one of the compartments of the pouch.

An additional aspect of the invention is a luggage article having a frame with an interior volume, a handle coupled to the frame, and material carried by the frame to enclose the interior volume. The material may be transparent such that substantially the entire interior volume is visible from outside the frame.

An additional aspect of the invention is a luggage article with a telescoping handle.

An additional aspect of the invention is a luggage article with least one wheel coupled to the frame.

An additional aspect of the invention is a luggage article having a body having an interior volume, the body being made of a transparent material such that substantially the entire interior volume is visible from outside the body. The body may have an opening to provide access to the interior volume and a handle and/or strap coupled to the body.

An additional aspect of the invention is a luggage article wherein the interior volume is sized and configured to receive a laptop computer.

### BRIEF DESCRIPTION OF THE DRAWINGS

FIG. 1 is an anatomic side section prior art view of a human abdomen showing an interior wound area and a tube that is placed according to conventional techniques to drain fluid from a seroma at the wound site.

FIG. 2 is a view showing a representative collection of the plurality of items that a patient having a post-operative drain desires or requires for their post-operative needs.

FIG. 3 shows a post-surgical accessory convenience kit, or PACK that includes the plurality of items shown in FIG. 2, as well as a carrier or pouch that includes a plurality of pockets or compartments that are sized and configured to hold the diverse array of items shown in FIG. 2.

FIGS. 4 and 5 show the placement of the items shown in FIG. 2 within the pouch shown in FIG. 3.

FIG. 6 shows the kit shown in FIG. 3 when in use, being worn about the waist of a patient.

FIG. 7 shows the kit shown in FIG. 3 when in use, being worn about the shoulder of a patient.

FIG. 8 is a front elevation view of an alternative embodiment of a post-surgical accessory convenience kit.

FIG. 9 is a rear elevation view of an alternative embodiment of the post-surgical accessory convenience kit of FIG. 8.

FIG. 10 is a left side elevation view of an alternative embodiment of a post-surgical accessory convenience kit of FIG. 8.

FIG. 11 is a right side elevation view of an alternative embodiment of a post-surgical accessory convenience kit of FIG. 8.

FIG. 12 is a top plan view of an alternative embodiment of a post-surgical accessory convenience kit of FIG. 8.

FIG. 13 is a bottom plan view of an alternative embodiment of a post-surgical accessory convenience kit of FIG. 8.

FIG. 14 is a perspective view of an embodiment of a see-through computer bag.

FIG. 15 is an embodiment of a see-through carry on bag.

FIG. 16 is an embodiment of a see-through suitcase.

FIG. 17 provides a further arrangement of the present invention.

FIG. 18 provides a flow sheet carrying out a post-operative regimen for a patient that utilizes the present invention.

FIG. 19 demonstrates the communication capabilities of the present invention, showing the invention capable of wireless transmission of information from the present invention to external devices.

FIG. 20 demonstrates the use of an external hard drive being used in connection with the present invention to transfer information to an external computer.

### DESCRIPTION OF REPRESENTATIVE EMBODIMENTS

Although the disclosure hereof is detailed and exact to enable those skilled in the art to practice the invention, the physical embodiments herein disclosed merely exemplify the invention that may be embodied in other specific structure. While the preferred embodiment has been described, the details may be changed without departing from the invention, which is defined by the claims.

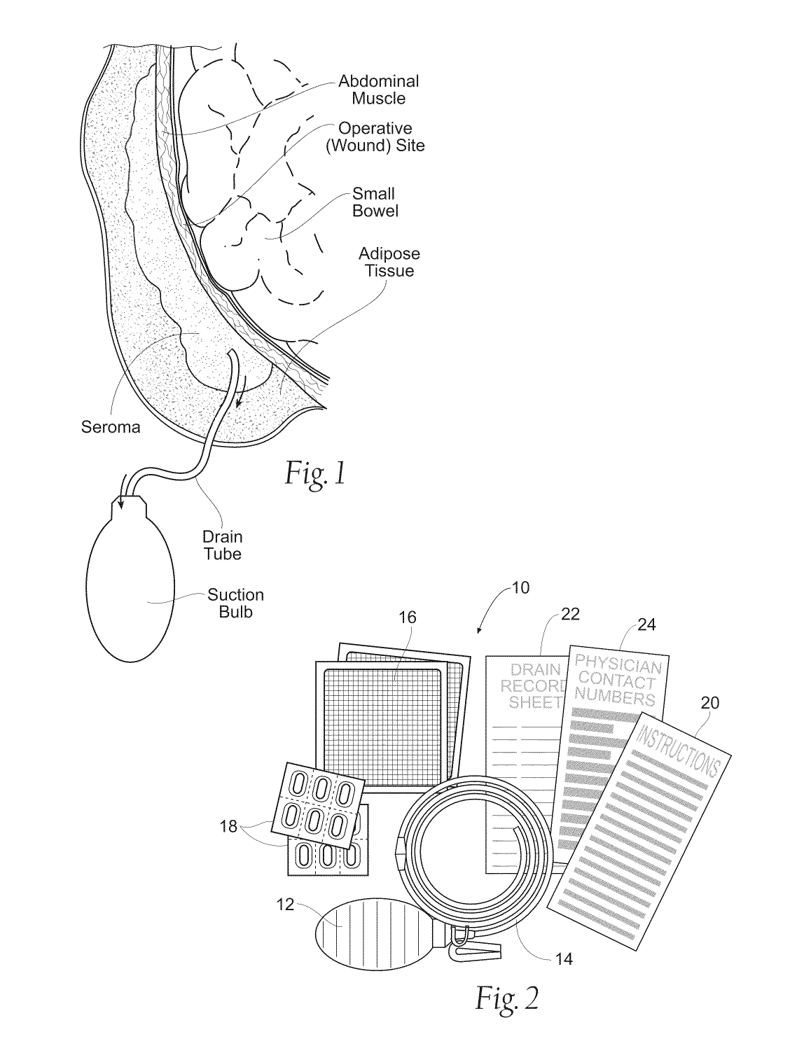

FIG. 2 shows a representative collection of the plurality of items 10 that a patient having a post-operative drain desires or requires for their post-operative needs. As shown in FIG. 2, the items 10 include, but not limited to, a drain suction bulb 12 (reservoir) and attendant tubing 14 (which, in use, is implanted at a drain site (as shown, e.g., in FIG. 1). The items 10 can further include dressing change materials 16, and post-operative medication 18 (e.g., pain medication, antibiotics, ointments, and other medication prescribed by a physician). The items 10 can further include physician or caregiver instructions 20, drain record sheets 22, and caregiver contact phone numbers 24.

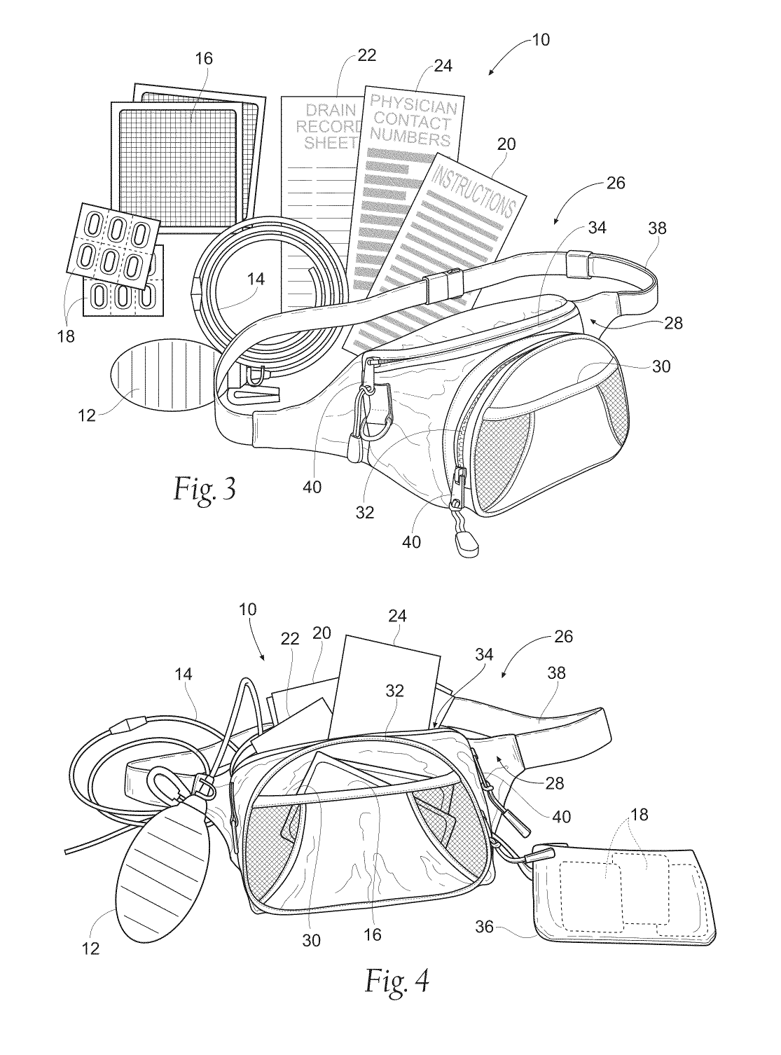

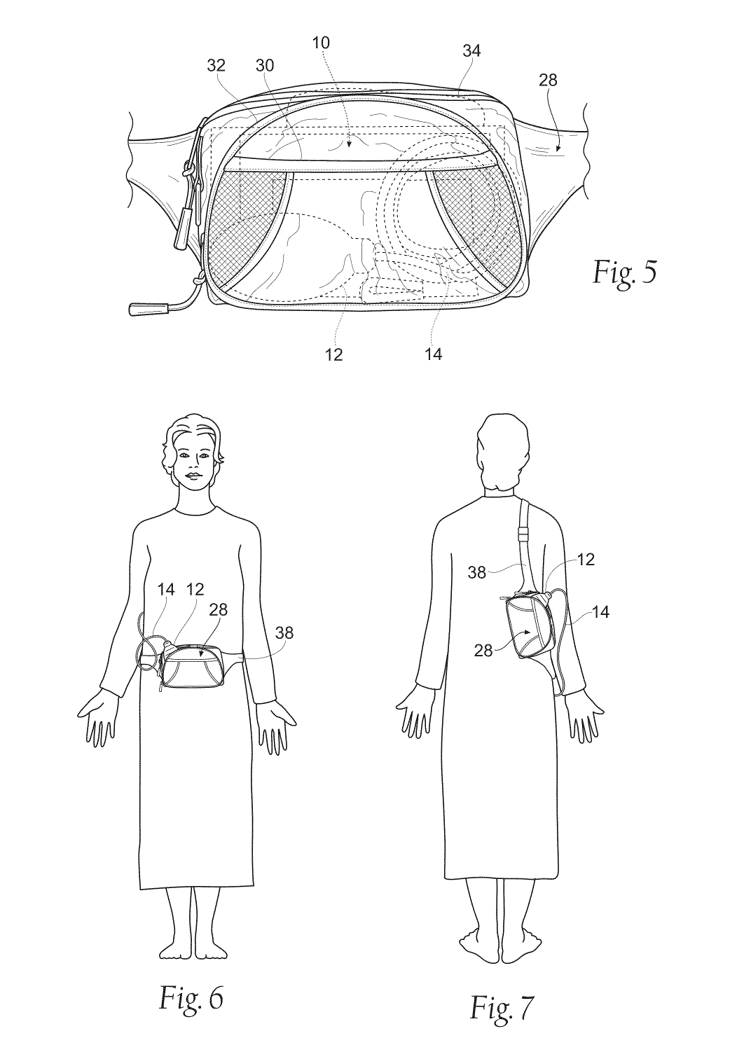

FIG. 3 shows a representative embodiment of a post-surgical accessory convenience kit 26, or PACK. The kit 26 includes all or some of the plurality of items 10 previously described and as shown in FIG. 2, which serve a patient's post-operative needs. As FIG. 3 shows, the kit 26 further includes a carrier or pouch 28. As shown in FIGS. 4 and 5, the pouch 28 includes one or more pockets 30; 32; 34 or attachable auxiliary cases 36 that are sized and configured to hold a number of the items 10 (such as shown in FIG. 2), which are directly related to the patient's post-operative needs. One or more auxiliary cases 36 can be attached by Velcro or carabineers on the external/outside surface of the pouch 28, as FIG. 4 shows. One or more of the pockets 30; 32; 34 and/or auxiliary cases 36 can be sized and configured to hold medication including pain medication, antibiotics and other medication, as well as dressings and dressing change materials. It should be understood that the kit of the present invention may include any combination of the items 10 listed above. It should be further understood that any additional item related to a patient's post-operative needs may be added to the kit.

The pouch 28 is desirably made of a material that is water and fluid resistant. The pouch includes a belt or strap 38 that makes it possible to secure the pouch 28 about the waist of a patient (see FIG. 6). The strap 38 could also be modified to allow the pouch 28 to be worn over the shoulder (see FIG. 7).

Some or all of the pockets 30; 32; 34 or attachable auxiliary cases 36 may include zippers or other suitable closure elements 40 (such as a slit or Velcro material). The closable pockets 30; 32; and 34 and auxiliary cases 36 can conveniently house surgical instructions, drain record sheets, and emergency physician and patient contact numbers, keeping these paper items protected from the elements and from loss. The pockets, when open, aid in the ease of removal of the contents out of the pocket.

At least one of the closable pockets 30; 34; 34 is desirably sized and configured to hold the drain suction bulb or reservoir 12 (which is coupled to the drain tubing 14 implanted in the patient), as well as other drain accessories. Placing the suction bulb 12 and the external length of tubing 14 within a pocket in the pouch 28 worn close to the incision and drain entry site can serve to relieve tension on the incision and drain entry site and avoid accidental pulling of the drain or suction bulb 12.

The pouch 28 can, if desired, be individualized with a patient's name, hospital or physician logos, or companies or practice names. After post-operative needs of the patient end, the pouch 28 can be cleaned, kept, and converted to everyday hiking or other non-medical uses.

The kit 26 may be supplied as a courtesy by caregivers to post-surgical patients, particularly those with surgical drains in place. For hospital based procedures, these would likely be covered by insurance. The kit 26 could also easily be incorporated into cosmetic surgeon's practices and given as a courtesy to patients post-operatively as a “bonus.”

Benefits of the kit 26 include convenience, comfort, accessibility, consolidation. The kit 26 makes it conveniently possible to have all desired or required post-operative items in one location, and the kit 26 goes where the patient goes. The kit 26 adds comfort by taking tension off of the incision and drain entry site and avoiding accidental pulling of the drain or suction bulb. The kit 26 is easily accessible and consolidates everything the patient needs with multiple pockets and locations for drain, dressings, and accessories. The kit 26 can be pre-packaged for each individual patient to hold Post-operative Instructions, Drainage Records, Contact numbers, Pain Medicine, Antibiotics, Other Medication, Antibiotic Ointment, Dressings, and, of course, the suction bulb for the drain.

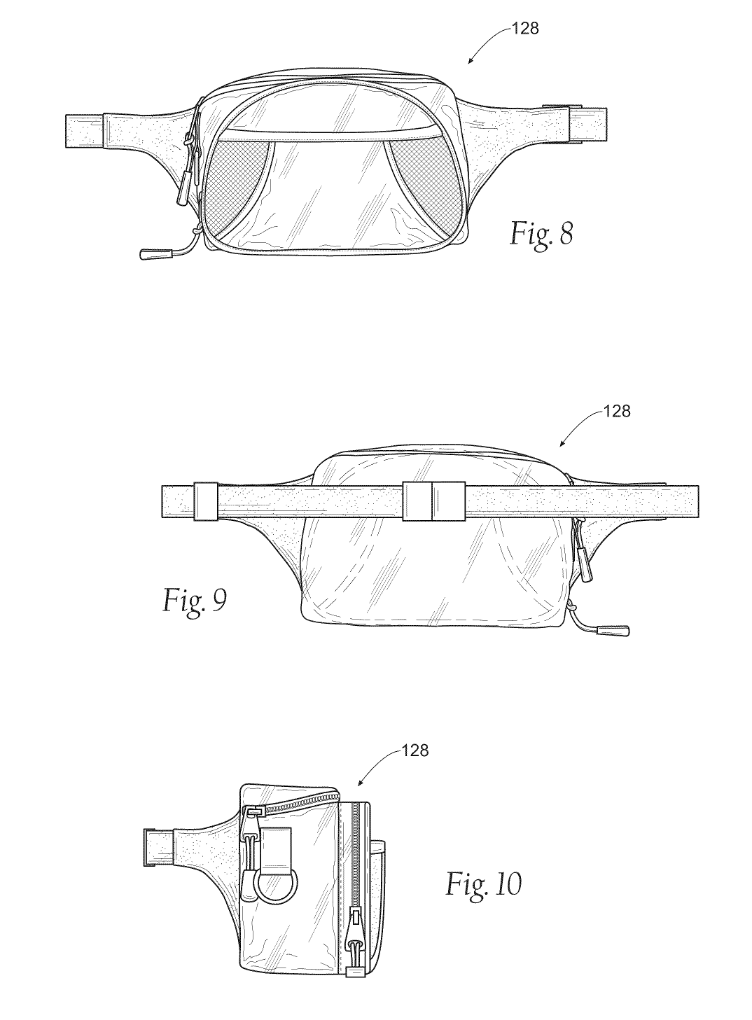

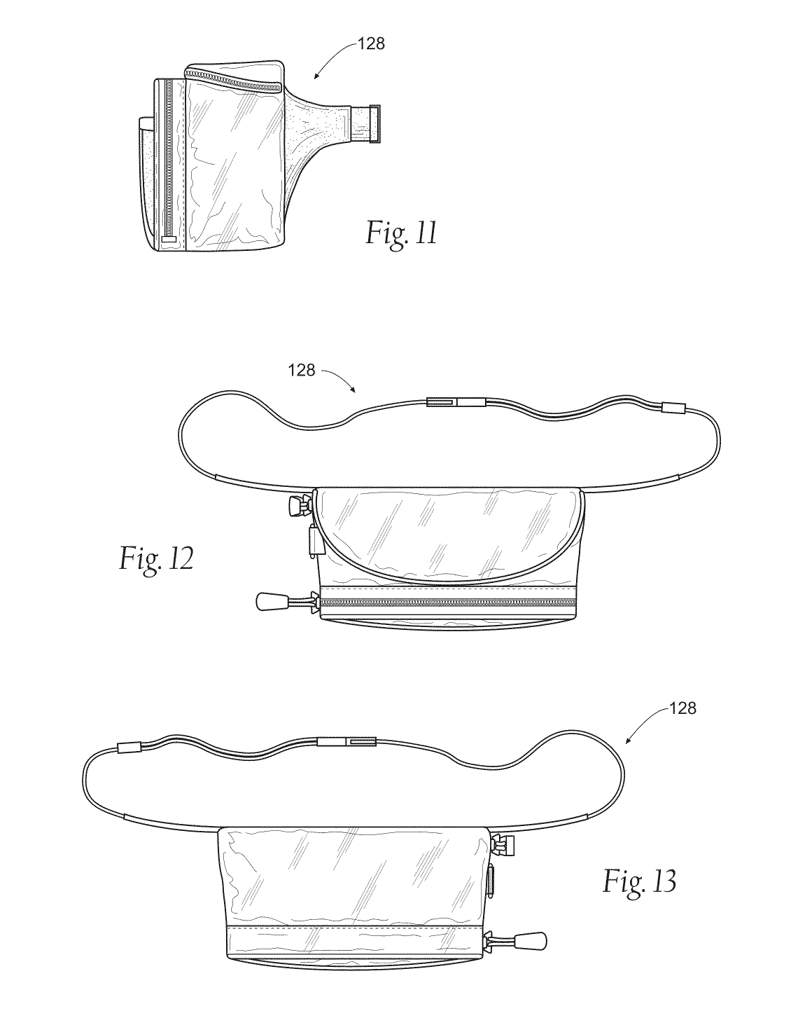

In an alternative embodiment of a pouch 128, as is shown in FIGS. 8 to 13, the pouch may be made of a see-through material. The see-through material may include, but is not limited to a clear plastic material, or a mesh metal or fabric. One or more of the items 10 shown in FIG. 2 may be placed within the pouch 128. In this manner the items 10 placed within the pouch 128 are visible from outside the pouch 128. Such a pouch 128 may be desirable in setting in which a patient must go through a security screening, such as at an airport. Often in a security screening an individual must have their bags sent through an x-ray machine or be hand searched. Removing the pouch 128 and sending the pouch 128 through an x-ray machine is not desirable to a patient with a drain. It may also be undesirable to have the pouch 128 hand searched. The see-through pouch allows security screeners to inspect the items 10 within the pouch 128 without removing the pouch 128 from the patient and without removing the items 10 from the pouch.

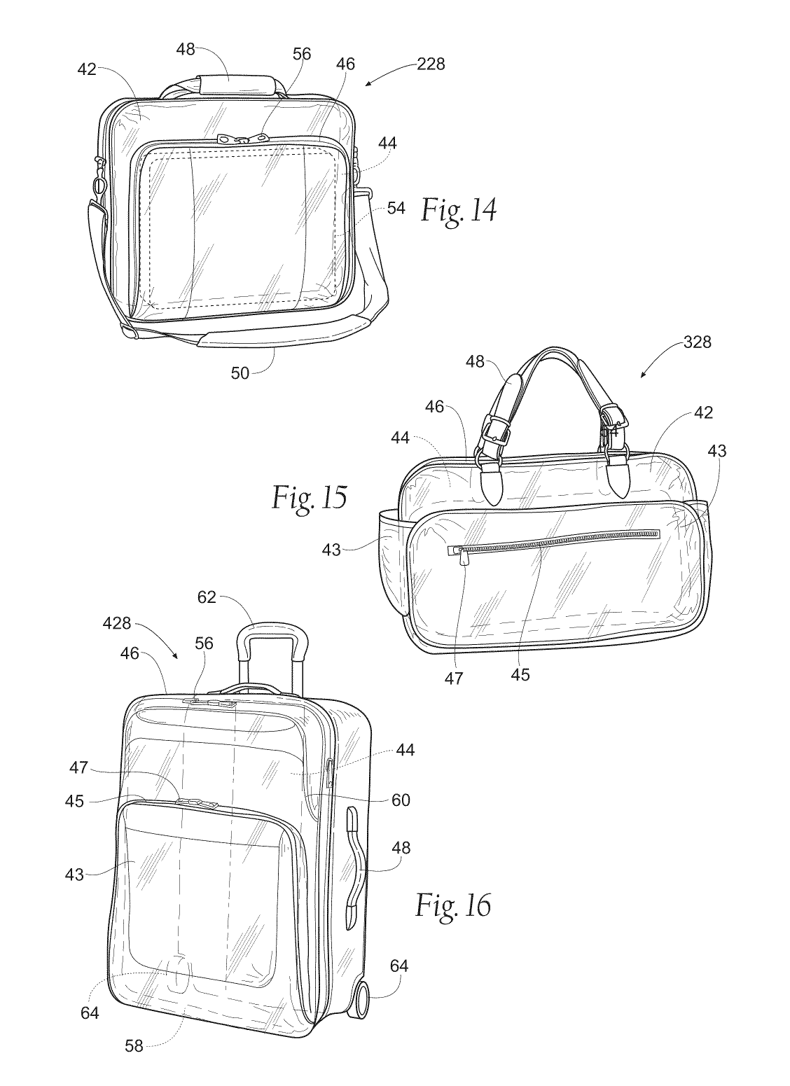

It is further contemplated that additional types of containers such as laptop cases or briefcases (see FIG. 14), carry-on bags (see FIG. 15), and suitcases (see FIG. 16) may be made of a see-through material. In this manner, the bag 228,328,428 may be inspected at a security checkpoint without sending the bag 228,328,428 through an x-ray machine or opening the bag 228,328,428 and searching the contents. As described above, the bag 228,328,428 may be made of any see-through material including, but not limited to a clear plastic material, or a mesh metal or fabric. It may be desirable to use such a bag 228,328,428 in any setting where security may be a concern and where it would be desirable to view the contents of a bag 228,328,428. For example, in a school, sports arena, airport, etc.

As shown in FIGS. 14 and 15, the bag 228,328 may include a body 42 having an interior volume 44. The body 42 may be made of a transparent material such that substantially the entire interior volume 44 is visible from outside the body 42. The bag 228,328 may include an opening 46 formed on the body to provide access to the interior volume 44. The bag 228,328 may include a handle 48 and/or a shoulder strap 50 coupled to the body 52. It is further contemplates that the interior volume 44 of the body 42 may be sized and configured to receive a laptop computer 54.

It is further contemplated that the interior volume 44 of the body 42 may be divided into a plurality of segregated storage compartments. It is contemplated that the segregated storage regions may be formed out of a transparent material. It is further contemplated that the bag 228,328 may include means for closure 56 of the interior volume 44. The means for closure 56 may include any means known in the art including, but not limited to a zipper or Velcro. The bag 228,328 may further include a flap which extends over the opening 46 to the interior volume 44. The bag 228,328 may further include at least one pocket 43 or storage compartment formed on one of the inside or the outside of the body 42. The pocket 43 may be formed of a transparent material. The pocket 43 preferably includes an opening 45 to access the interior of the pocket. The pocket may further include closure means 47, including but not limited to Velcro or a zipper, to close the pocket.

As seen in FIG. 16, the bag 428 may include a frame 58 having an interior volume 44 and a handle 48 coupled to the frame 58. The bag 428 may further include material 60 carried by the frame 58 to enclose the interior volume 44. The material 60 may be transparent such that substantially the entire interior volume 44 is visible from outside the frame 58.

The bag 428 may further include a telescoping handle 62. The bag 428 may further include at least one wheel 64 coupled to the frame 58. The bag 428 may further include an opening 46 on the bag 428 to provide access to the interior volume 44. It is further contemplated that the bag 428 may include means for closure 56 of the interior volume 44. The means for closure 56 may include any means known in the art including, but not limited to a zipper or Velcro.

The bag 428 may include a storage compartment sized and configured to receive a laptop computer. The storage compartment may be made of a transparent material. The bag 428 may further include at least one pocket 43 or storage compartment formed on one of the inside or the outside of the body 42. The pocket 43 may be formed of a transparent material. The pocket 43 preferably includes an opening 45 to access the interior of the pocket. The pocket 43 may further include closure means 47, including but not limited to Velcro or a zipper, to close the pocket.

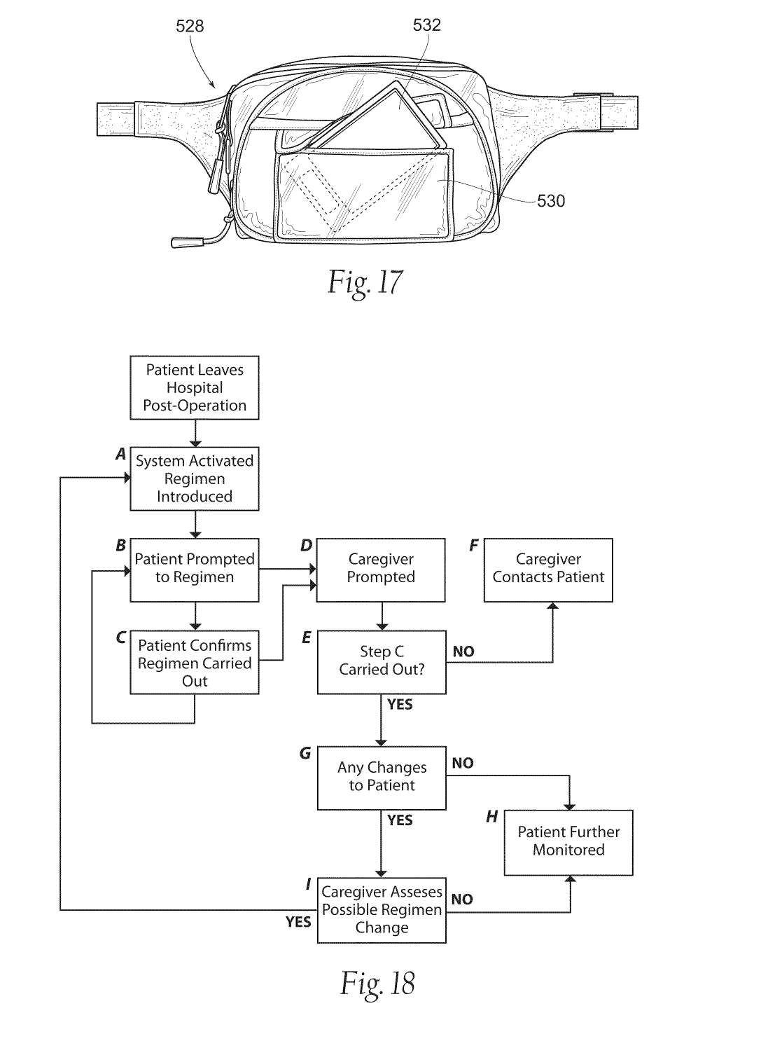

FIG. 17 demonstrates the further adaptability of the present invention. The pouch 528 is designed similarly to the previously discussed pouches, 28, 128, so that the pouch 528 may be worn and transported easily by the patient. Likewise, the pouch 528 comprises a plurality of compartments for retaining and holding various items, as previously described with respect to FIGS. 1-4.

The pouch 528 also provides a compartment 530 for storage of an information transmission device (ITD) 532. The ITD 532 allows for transmission of information to and from a caregiver related to the post-operative procedures and regimens that the patient is to follow. The ITD 532 allows for the patient's post-operative regimen to be monitored by the patient, as well as allowing for the potential ability for a caregiver to monitor to monitor and adapt the regimen as necessary.

In practice, the ITD 532 will be personalized to monitor various characteristics of the patient, such as times and quantities of medicine taken, when to change bandages and dressings, the color and amount of discharge coming from a wound, and other characteristics, such as the patient's temperature or other vital characteristics. The regimen that ITD employs will be specifically designed towards a particular patient's specific needs.

The ITD preferably is a software based device that has the ability to communicate remotely to a caregiver. Examples of such devices include computer applications that can be used with Smart phones, MP3 players, or similar devices, e.g. wireless devices, that allow for the retention and communication of information remotely to and from the caregiver, and, also, the ability for the treatment regimen to be updated.

FIG. 18 provides a schematic for the use of the device described with respect to FIG. 17. After an operation, the patient will leave the hospital. The ITD 532 will be activated in step A, with the post-operative instructions for the patient entered into the ITD 532. In step B, the patient is prompted to carry out the instructions specific to that patient, e.g. take a specific dosage of medicine or change the patient's dressing. At step C, the patient confirms that the instructions at step B were carried out. The patient may also be prompted to enter other information, such as the color or the discharge around the wound, the patient's temperature, or other vital signs of the patient, as is relevant to that particular patient. Once step C is complete, the ITD 532 will process the information and reset so that the patient will again be prompted at the next interval to complete step B.

The ITD 532 prompting at step B will also provide a prompt to the caregiver at step D. This will allow the caregiver to monitor the patient. The caregiver will also be notified when step C has been completed. At step E, if step C has not been carried out by the patient, the system will prompt the caregiver to contact the patient at step F to determine why step C was not carried out. The times between steps B and C and the notification of step F can be adjusted according to a patient's specific needs and procedure.

If the caregiver has been prompted that steps B and C have been carried out, the caregiver will review the information submitted by the patient at step C and determine if there are any changes to the patient or the patient's injury. If there are no changes to the patient, the caregiver can note this and further monitor the patient at step H.

If there are changes to the patient, the caregiver will address these changes at step I. The changes may be the normal healing process, in which case the caregiver will further monitor the patient at step H. However, the changes may be important enough that the caregiver may decide to change or alter the patient's regimen. For example, if the patient is noticing a particular color of discharge around a wound, and the patient transmits this information at step C, the caregiver may provide instructions to alter the regimen at step A, e.g. the regimen may be adjusted to change the patient's dressing more frequently. Similarly, if the patient is experiencing an unexpected fever, the caregiver may instruct a change in the medication that the patient is taking. The caregiver will be able to transmit these changes, either as a prompt to the patient at step A and/or as an update to the software program used at step A.

As stated above, one of the advantages of the present invention over prior art systems is the ability for the regimen to be adapted to a patient's particular needs, as well as adapting the regiment while being carried out by the patient. The ITD 532 can also be programmed for a particular patient and the patient's needs and comfort levels. For example, response to lack of prompting at Step D may be set to take place relatively quickly, e.g. a few hours, after failure of step C to be carried out, or the prompt may take place after a longer period of time, e.g. 1 day. Likewise, the caregiver may be notified only after several iterations of the regimen have or have not been carried out, or the information of steps B and C may be stored and retrieved by the caregiver at a follow-up checkup or appointment. By allowing the ITD 532 to be programmed for the particular needs and uses of a patient, the patient can be more efficiently treated and monitored.

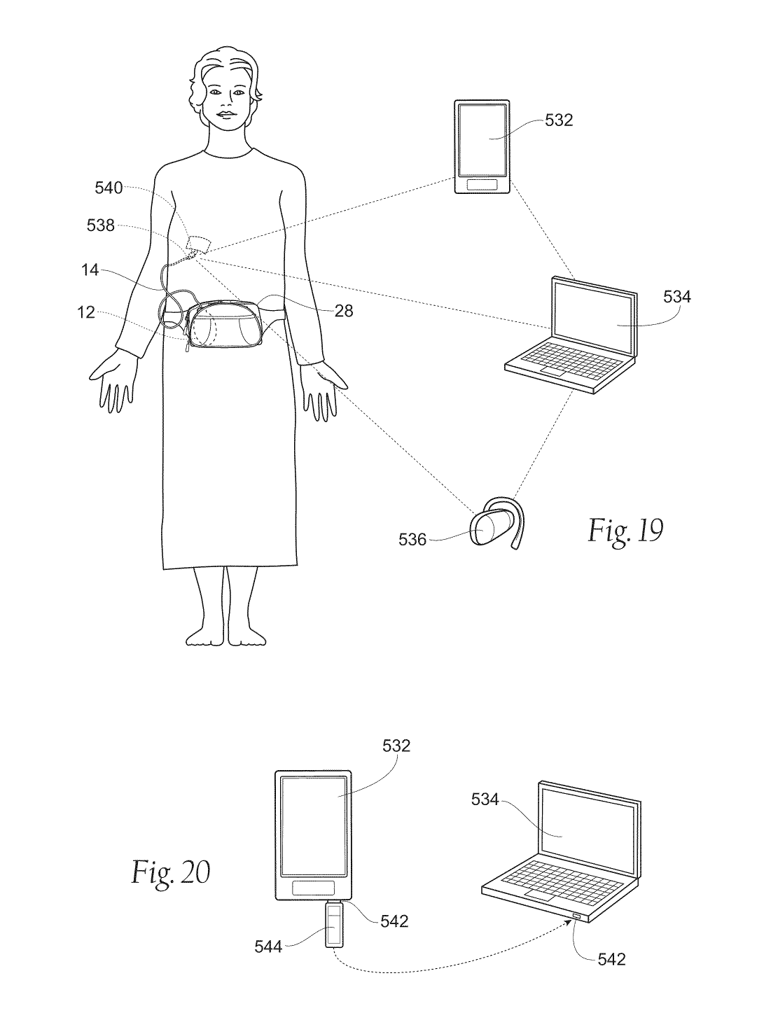

The present invention, as depicted in FIG. 19, has the capability of communicating information to several various external devices, such as the ITD 532, which can include devices such as an iPhone™, iPad™, Droid™, Blackberry™ smart phones, MP3 player, PDA, and other similar devices, a computer 534, or a Bluetooth™ device 536 or other similar wireless devices. These devices can communicate between one another and can be synchronized, as well, to insure that the patient's information is properly communicated between the patient and the caregiver. Likewise, the use of wireless transmission of information allows the instantaneous downloading and updating of information to a computer or other data storing medium.

FIG. 19 also shows that the drain tube 538 may communicated directly with the external devices, discussed above. For example, the drain tube 538 may include a transmission device 540, e.g. a microchip or radio frequency identification (RFID) device, that will allow transmission of information directly to the external device. For example, it may be advantageous to transmit information directly related to the drain tube 538, e.g. dislodgement of the drain tube port or an improper connection of the drain tube. Such an arrangement may include an alarm, as well, to notify the patient and/or the caregiver if there is a change in the connection of the drain tube 538.

The ability to transmit information with the use of the present invention is also demonstrated in FIG. 20. The ITD 532 may have a USB port 532, which allows the attachment of an external hard drive 544, e.g. a zip drive, which will allow the transfer of data from the ITD to the computer 534. The ability to transfer data as shown further demonstrates the utility and advantages of the present invention in monitoring a patient's post operative conditions, as well as allowing regimens to be adapted quickly and efficiently in the event of a patient's changing conditions or status. This also provides an example of how the patient's information and regimen may be updated by the caregiver.

The present invention is useful for patients that have or do not have a wound drain as part of their post-operative recovery regimen. The ability to tailor a regimen particular to a patient and for the ITD 532 to monitor the patient's regimen can be performed for post-operative situations, in general. The ability to notify a patient when to take a medicine, change a dressing, etc., can be used to benefit all patients, regardless of whether the patient has a wound drain or not. Similarly, the ability of the present invention to provide alarms and warning for both the patient and the care giver greatly improves post-operative care.

The foregoing is considered as illustrative of the principles and technical features of the invention. Furthermore, since numerous modifications and changes will readily occur to those skilled in the art, it is not desired to limit the invention to the exact construction and operation shown and described. While the preferred embodiment has been described, the details may be changed without departing from the principles and technical features of the invention.

I/We claim:

1. A post-operative kit for use by a patient comprising:
an information transmission device (ITD) for communicating post-operative regimen information specifically directed to the patient.

2. The kit of claim 1 further comprising a pouch for storing the ITD.

3. The kit according to claim 1 further comprising at least one dressing change material.

4. The kit according to claim 1 further comprising at least one post-operative medication.

5. The kit according to claim 3 wherein said at least one post-operative medication includes at least one of a pain medication, an antibiotic, and an ointment.

6. The kit according to claim 1 wherein said ITD device is capable of remotely transmitting information to the patient and to a care giver.

7. The kit of claim 1 wherein the ITD device is capable of updating the regimen information.

8. A post-operative device for use by a patient comprising:
an information transmission device (ITD) for communicating post-operative regimen information specifically directed to the patient.

9. The device of claim 8 wherein the ITD is capable of remotely transmitting information to the patient and to a care giver.

10. The device of claim 8 wherein the ITD is capable of updating the regimen for the patient.

11. The device of claim 8 further comprising
an alarm for informing the patient and/or the care giver when the regimen has not been carried out.

12. A method of monitoring a patient in a post-operative comprising the steps of:
providing an information transmission device (ITD) for the patient; and
programming the ITD to carry out a regimen specifically directed to the patient.

13. The method of claim 12 further comprising the steps of:
communicating the information from the ITD remotely to a care giver.

14. The method of claim 13 further comprising the step of:
adjusting the regimen based on the information communicated to the care giver.

15. The method of claim 12 further comprising the step of alerting the patient if a portion of the regimen is not followed.
